(19) United States
(12) Patent Application Publication
(10) Pub. No.: US 2018/0194501 A1
(43) Pub. Date: Jul. 12, 2018

(54) Method for Sterilizing Unpackaged Consumable Items and Storage Containers and Outer Package Used for This

(71) Applicant: SIG Technology AG, Neuhausen am Rheinfall (CH)

(72) Inventors: Hans-Willi Mainz, Heinsberg (DE); Michael Schreder, Hochfilzen (AT)

(21) Appl. No.: 15/746,156

(22) PCT Filed: Jul. 20, 2016
(86) PCT No.: PCT/EP2016/067243

§ 371 Date: Jan. 19, 2018

(30) Foreign Application Priority Data
Jul. 23, 2015 (DE) 10 2015 112 034.8
Sep. 30, 2015 (DE) 10 2015 116 536.8

(51) Int. Cl. B65B 55/10; B65B 5/06; A61L 2/20

(52) U.S. Cl. CPC B65B 55/10; A61L 2/208; B65B 5/061

(57) ABSTRACT

A method for sterilising unpackaged consumable items by means of a treatment agent having a disinfectant, and using storage containers and outer packaging. In order that the abovementioned method can be designed and further developed so that contamination of the consumable items is reliably excluded, a certain quantity of unpackaged consumable items is stored together for a specified time in a storage container and the inside of the storage container is treated with a predetermined quantity of the treatment agent. In order to achieve “long-term sterilisation” a storage container serves to accommodate a plurality of unarranged, unpackaged consumable items, with the material of the storage container being plastic, wherein the storage container is designed as a bag, which after filling with consumable items and subsequent treatment is closed and then stored in a closed outer package.

The invention first of all relates to a method for sterilising unpackaged consumable items, such as in particular plastic opening and/or closing elements for subsequent use in beverage or food packaging by means of a treatment agent having a disinfectant. In addition, a storage container to accommodate a plurality of unarranged consumable items and an outer package to accommodate one or more such container/s are claimed.

The nature of the consumable items to be sterilised is extremely varied. The invention is aimed in particular at those consumable items, that concern packaging or parts of packaging for food or the medical field. The invention is explained in more detail in the following using as an example in particular plastic opening and/or closing elements for subsequent use in food or beverage packaging.

Where the following relates to opening and/or closing elements for beverage or food packaging, this is not intended to cover just the most widely-used re-sealable pouring elements, but any kind of opening aids and/or closures for such containers.

The aseptic filling of beverages or foods has always been subject to the highest standards of sterility. Thus, it has been known for a long time to sterilise the corresponding packaging and also the associated additional elements coming into contact (at the latest upon opening) with the product, such as opening elements, closures or pouring elements, wherein the sterilisation usually takes place immediately before the filling process and final sealing of the packaging.

Filling is generally performed on what are known as filling machines, located at the filling plant and the receptacles to be filled must be transported from their place of manufacture to the filling plant site.

From WO 2012/00573 A1 a device is known for sterilising receptacle closures, in which a plurality of cap-like closures are transported through several treatment zones and sterilised there though the application of H₂O₂ vapour. Here the sterilisation takes place by direct application to the opening and/or closing elements of disinfectant and a subsequent drying process, in order to reliably remove the H₂O₂ again.

From DE 40 31 472 A1 a device is in itself known for sterilising, filling and sealing a container having a filler opening.

DE 197 27 942 A1 describes a machine and a method for sealing bottles with caps having a corresponding sterilisation device.

The sterilisation of the opening and/or closing elements usually takes place immediately before they are attached to the receptacle to be filled, for example as is known from DE 10 2005 032 322 A1.

Since the common opening and/or closing elements are generally injection moulded, their manufacture may take place at a different location, meaning that these elements also have to be transported to the filling plant.

Following manufacture and in particular when in transit, the individual elements are always in danger of being exposed to contamination by vegetative microorganisms.

Vegetative microorganisms are understood to be unicellular organisms able to multiply by cell division, likely to multiply in the contents (‘product’) of a package and in so doing alter the contents. The term also covers the surviving forms of the unicellular organisms able to multiply, such as for example their spores.

These spores are usually highly resistant to changes in their surrounding environmental conditions. If microorganisms are unable to find an environment in which to metabolise and/or reproduce, some microorganisms have the possibility of switching to a spore stage.

More precisely in the context of this application, the term microorganisms is intended to mean eukaryotes and prokaryotes, wherein the eukaryotes have a true cell wall and comprise algae, protozoa, fungi and slime mould, while prokaryotes cover the group of bacteria (see ‘Bergey's Manual of Determinative Bacteriology’, 8th Edition, Baltimore: Williams & Wilkins, 1974).

With prokaryotes in particular surviving forms are known such as for example spores. An increase in these can be found for example, even after thermal or chemical treatment of raw materials for the manufacture of raw cardboard products in the latter because such treatment methods either kill the form of the microorganisms directly capable of multiplying or initiate switching to the spore form.

As a measure of the number or quantity of microorganisms contained in an amount of substance (for example in said raw cardboard product), the person skilled in the art will be aware of the term colony forming units per unit of weight or area (CFU/g) or (CFU/cm²). Unlike the direct counting of all microorganisms present using suitable optical means, the determination of the number of colony forming units takes place by means of the specific multiplication of the microorganisms capable of dividing under suitable culture conditions. This generally takes place up to a colony size that can be counted with the unsupported eye. Here use is made of the fact that from each individual microorganism capable of dividing under predefined conditions, precisely one colony results. Individual cases in which two CFU are so close together that only one visible colony is formed from them are then generally ignored.

Typical microbiological determination method standards are provided in ISO 8784-1 from 2005.

A reduction in the CFU/g or CFU/cm² will accordingly be used by the person skilled in the art as a measure of the effectiveness of a method of germ reduction and is often referred to as the disinfection rate. The sterility rate for the number of packages produced is derived from this. For aseptically packaged foods it has frequently been the case here that as a maximum the contents of one in every 3,000 packages produced should be unsterile over the period up to the best before date, generally 12 months. More recent efforts by the applicant now aim for a maximum of one package for every 30,000 or even 50,000 packages produced.

From DE 10 2011 111523 A1 it is also already known to treat the top or bottom open cut edges of a carton sleeve of a packaging material by the application of a treatment agent containing a disinfectant, wherein the disinfectant remains on the cut edges following application and penetrates the packaging material. The application here takes place by spraying from above, wherein a plurality of flat-folded packaging sleeves are stacked together.

Trials have shown that the idea of the long-term effect of treatment agent on the item to be treated during the transport time already available, is transferrable not only to fibrous packaging material, but also to the flat surfaces of other unpackaged consumable items such as for example of opening and/or closing elements. This is in particular of considerable interest if the opening and/or closing elements are not only to be applied to a package, but actually form part of a package, for example the head section of a cardboard/plastic composite packaging, wherein the actual pouring opening constitutes only a small part of the complete plastic injection moulded head section, and therefore at least in those cases in which areas of the opening and/or closing elements come into contact with the product contained in the package before the package is opened.

From U.S. Pat. No. 9,078,435 A1 a method is known for sterilisation of unpackaged containers, in which the subsequently hygienically used container, has a treatment agent having a disinfectant and a reaction accelerator placed in it and this is closed tight or sealed for a long time from a number of minutes to a number of days and the internal wall surfaces of the container are thereby disinfected. In this way the seal that seals the container is also disinfected. The teaching of U.S. Pat. No. 9,078,435 A1 is aimed here at the disinfection of what are known as bag-in-box bags which, due to their subsequent use, must have very high barrier characteristics, and thus very low water- and gas-permeability. Since the individual container, that is the “bag-in-box” bag, in order to achieve its subsequent intended purpose should only have a small quantity of treatment agent, however, in the disclosed method an extremely low concentration of disinfectant is used. Here the use of extremely low concentrations of disinfectant, preferably H₂O₂, is also necessary due to the damage that would otherwise be caused to the container walls coming into direct contact with the treatment agent.

On this basis, the problem for the present invention is to arrange and develop the method mentioned and described above for sterilising unpackaged consumable items, such as in particular plastic opening and/or closing elements for subsequent use with beverage or food packaging, by means of a treatment agent having a disinfectant so that contamination of the consumable items with germ-forming spores is reliably excluded. It is particularly desired to achieve a “long-term sterilisation”, in which the sterilisation of the treated consumable items takes place prior to their final use. For the example of the opening and/or closing elements this means that the sterilisation of these should be completed before the opening and/or closing elements are introduced into a filling machine. If the filling machine has an aseptic area, within the filling machine further disinfection of the opening and/or closing elements can take place.

This problem is solved by a method according to the preamble of Claim 1 in that a certain quantity of unpackaged consumable items is stored for a predetermined time in a storage container and in that the inside of the storage container is treated with a predetermined quantity of the treatment agent. The method can also be referred to as ‘immediate indirect sterilisation’. The storage container itself is not considered a consumable item for the purposes of the present specification.

A corresponding storage container having the features of the preamble of Claim 14 solves the problem in that the material for the storage container is plastic, wherein the storage container is designed as a bag which after filling with unpackaged consumable items and subsequent treatment is sealed and stored in a subsequently sealed outer packaging.

An outer package according to the preamble of Claim 20 that can be used for this purpose, in order to solve the problem, is characterised in that cardboard, in particular corrugated cardboard, is used as material for the outer packaging, so that an outer carton with closable flaps results, which are folded down and suitably secured in order to close the outer carton. This allows secure transport of the treated consumable items as far as the place of use.

According to a further teaching of the invention the treatment of the individual opening and/or closing elements is performed mechanically, wherein preferably a spray nozzle is used. Spraying preferably takes place without air, wherein the storage container should then be immediately closed. It is also conceivable, however, for the treatment to be performed manually. In any event, the treatment should as far as possible take place immediately after manufacture of the consumable items, and in the example therefore after the cooling of the opening and closing elements following the injection moulding process, in order to avoid contamination from the outset.

According to a further preferred teaching of the invention, the treatment agent used is hydrogen peroxide (H₂O₂) and the concentration of the treatment agent is between 20% and 50%, preferably 35%.

Another preferred form provides that the amount of the respective treatment with the treatment agent is, depending on the volume and size of the surface of the consumable items to be sterilised, between 0.5 ml and 2 ml. The actual quantity required here can be easily determined by comparative trials.

In the example of the opening and/or closing element it was found that a dosage of one millilitre is sufficient for sterilising 1,200 opening and/or closing elements. This “standard dosing” is matched to the volume ratio of the standard carton size of the outer packaging, which in the example is 56 litres. The overall proportion of the volume of the opening and/or closing elements here is 14 litres, so that the maximum free air volume is 42 litres. “Standard dosing” means a normal dosing, since an overdose leads to the formation of condensate in the storage container, which must be avoided at all costs and an underdose is insufficient to reliably sterilise all the consumable items contained in the storage container. Obviously, the dosing quantity used must be adapted for differing volumes.

The normal dosing is approximately 1 ml/56 l. A deviation of + or −25% is still within the normal dosing range, however. The proportion of the volume of the consumable items here is ¼ of that of the storage container or the volume enclosed by the outer carton.

The duration of the exposure to the treatment agent in the storage container is preferably between 12 hours and 21 days, particularly preferably it is between 24 hours and 7 days.

This ensures that in the time taken for transport between leaving the manufacturer and arrival at the customer's full sterilisation has taken place, so that the, for example, opening and/or closing elements can be introduced more or less immediately after being unpacked from the storage container into the aseptic area of a filling machine.

The method is preferably actually free from active removal of the treatment agent. This means that the treatment agent is not actively removed from the sterilised unpackaged consumable items, such as in particular plastic opening and/or closing elements for subsequent use with drink or food packaging. While the removal without residue of disinfectants on areas of food packaging in contact with the product is a condition that must be met, it has surprisingly been found that the treatment agent containing disinfectant fully diffuses within the exposure time and the remaining residues fall below the legally permitted levels even on relatively smooth surfaces. The treatment agent therefore remains, as it were, on the treated consumable items, but its amount and effect fall over the stated time to such an extent that the requirements concerning contamination by treatment agents of surfaces of consumable items in contact with food can easily be met.

It is a major advantage if the treatment agent is free from a reaction accelerator. It may at first seem illogical for the low quantity of disinfectant used not to contain a reaction accelerator, in the way that, for example, formic acid acts for H₂O₂. But it has surprisingly been shown that the atmosphere that forms is sufficient to reliably sterilise the consumable items exposed to it, even if a reaction accelerator is dispensed with. Furthermore, the sterilising atmosphere can also be effective over a long period. Reaction accelerators are in fact unstable and have a maximum effectiveness of 12 hours. Once the disinfectant and the reaction accelerator come into contact in the treatment agent or even for the first time in the sterilising atmosphere that forms, the period of effectiveness of the disinfectants is also limited to the period of effectiveness of the reaction accelerator. In the example in which the disinfectant is formed by H₂O₂ or an aqueous solution of this and the reaction accelerator contains or consists of formic acid, the actual period of effectiveness is limited to a maximum of 12 hours. It has been shown, on the other hand, that the reducing capability of the sterilising atmosphere settling on the consumable items is much better when such a reaction accelerator is dispensed with.

A further form of the invention provides that a treatment agent is used that contains a dye and causes a discoloration of the treated storage container, so that it is optically detectable and if necessary it can be checked mechanically that all filled and treated storage containers have been treated with treatment agent.

A further form of the invention provides that the specified quantity of consumable items is measured in individual parts and/or individual modules and that the quantity of individual parts and/or individual modules is ≥100, in particular ≥500, quite particularly ≥800.

In this way the method can be applied particularly effectively. It has surprisingly been shown here that the individual parts, in particular the individual opening and/or closing elements, can be fully sterilised even in the stated quantity ranges. Tests have actually demonstrated that the number of individual parts can be even higher. Depending on the volume of the storage container of approximately 56 litres, the 1,200-1,500 individual parts, in particular individual opening and/or closing elements, that are normal these days in food packaging, in particular aseptically manufactured beverage packaging, with package volumes of between approximately 100 ml and approximately 2,000 ml, can be sterilised.

To allow high productivity to be achieved, it is envisaged that the method is free from positioning of the consumable item or consumable items. The consumable items are for example filled together as bulk goods in the storage container or arrive directly or indirectly at the exit of a production machine in the storage container.

In order to avoid condensation of treatment agent occurring on the inside of the storage container, according to a further form of the invention it is provided that the plastic material of the storage container has a predetermined permeability.

A further teaching of the invention provides that as the storage containers, plastic containers in polyethylene (PE) or polyamide (PA) are used.

Instead of spraying the consumable items in a further development of the invention it is also possible to introduce a treatment carrier, for example a strip of felt, treated with a sufficient quantity of sterilisation agent, into the storage container.

Moreover, it is also possible to further wet the outer package used on its inside with treatment agent, in order to reliably prevent, inside the—sealed—outer carton germ-forming spores reaching the storage container(s) from the material thereof.

Finally, it is also possible, to provide the outer packaging on its inside with one or more treatment carriers, in order to achieve the desired long-term sterilisation during transport. To this end the treatment carriers can be inserted or bonded in the outer carton.

The invention is explained in more detail in the following using a drawing showing a merely preferred embodiment. The drawing shows as follows in

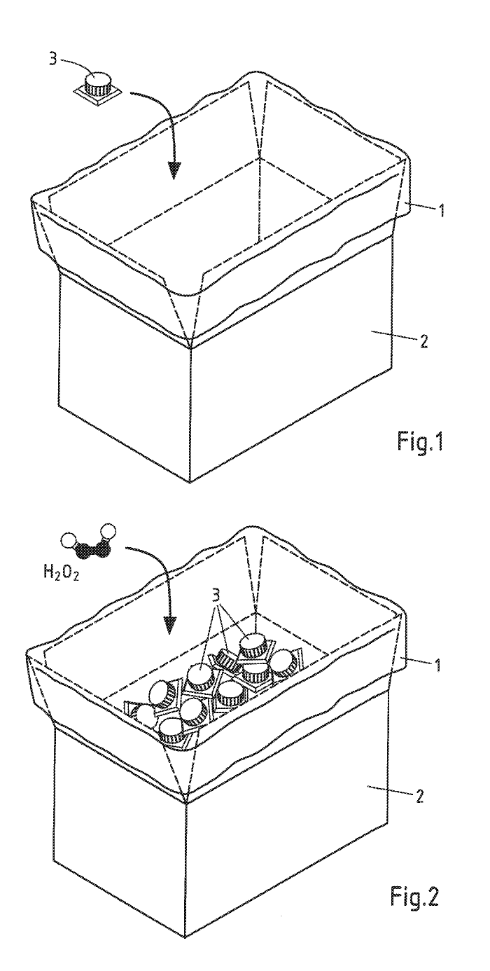

FIG. 1 a perspective view of an empty storage container in an outer packaging;

FIG. 2 the filled storage container from FIG. 1;

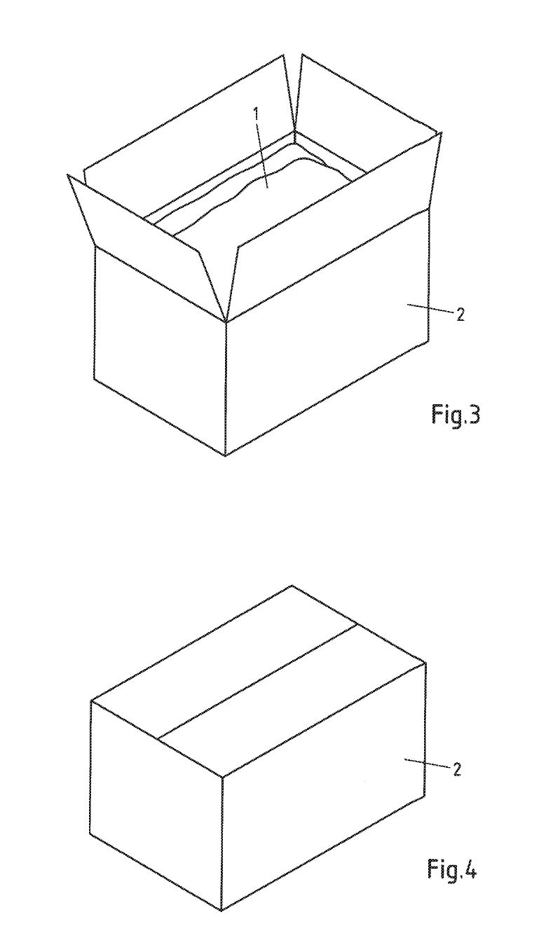

FIG. 3 the closed storage container from FIG. 2 in the open outer packaging;

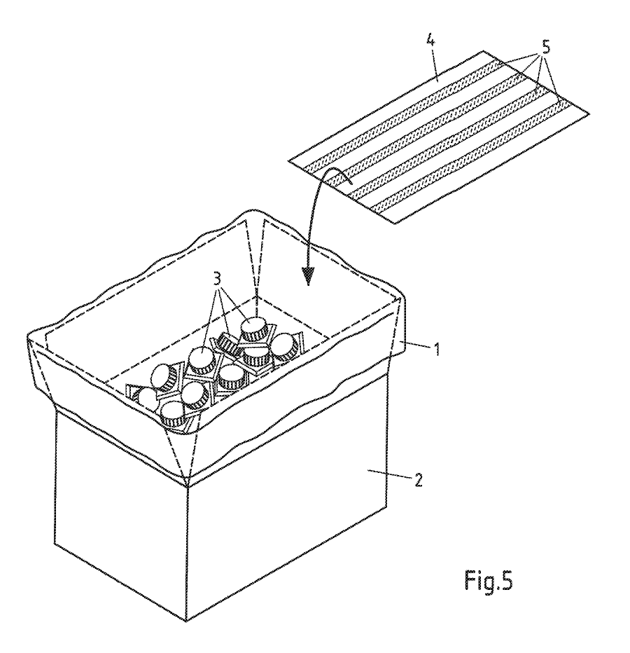

FIG. 4 the closed outer package; and

FIG. 5 a further embodiment concerning application of the sterilisation agent.

FIG. 1 shows a storage container 1 designed as a plastic bag, inserted in an open outer carton 2 so that its top edge can be guided over the flaps of the outer carton 2. In this way contact between the consumable items 3 to be filled into the storage container 1 and the outer carton can be avoided. As consumable items by way of example opening and/or closing elements for subsequent use with beverage or food packaging are shown.

FIG. 2 shows the storage container 1 filled with the abovementioned consumable items 3, in which for sterilisation purposes a predetermined quantity of the treatment agent H₂O₂ is introduced. The storage container 1 is then closed, as shown in FIG. 3 and the outer carton 2 is finally closed as shown in FIG. 4.

Finally, FIG. 5 shows a further possibility for introducing the sterilisation agent, in which this takes place by means of an inserted sheet 4, to which various treatment carriers 5, treated with a sterilisation agent, have been applied.

1.-22. (canceled)

23. A method for sterilising packaged or partially packaged or partially sealed consumable items by means of a treatment agent having a disinfectant, comprising the steps of sterilizing a consumable item or an area of a consumable item, disposing in a first sealed environment such that a plurality of individually packaged consumable items or sealed areas of consumable items are stored together for a specified time in a container, and treating the inside of the container with a predetermined quantity of the treatment agent by a treatment carrier, treated with a sterilisation agent, being introduced into the container.

24. The method according to claim 23, wherein the treatment is performed mechanically.

25. The method according to claim 23, wherein the treatment is performed manually.

26. The method according to claim 23, wherein the treatment agent hydrogen peroxide (H₂O₂) is used and wherein the concentration of the treatment agent is between 20% and 50%.

27. The method according to claim 26, wherein the concentration of the treatment agent is 35%.

28. The method according to claim 23, wherein the amount of the respective treatment with the treatment agent is, dependent upon the volume and the size of a surface of the consumable items, between 0.5 ml and 2 ml.

29. The method according to claim 23, wherein the duration of the exposure to the treatment agent in the container is between 12 hours and 21 days.

30. The method according to claim 29, wherein the duration of the exposure to the treatment agent in the container is between 24 hours and 7 days.

31. The method according to claim 23, wherein the method is free from active removal of the treatment agent.

32. The method according to claim 23, wherein the treatment agent used is free from a reaction accelerator.

33. The method according to claim 23, wherein the treatment agent used contains a dye and brings about a discoloration of the container.

34. The method according to claim 23, wherein a specified quantity of consumable items is measured in individual parts and/or individual modules and wherein the quantity of individual parts and/or individual modules is ≥100.

35. The method according to claim 23, wherein the method is free from positioning of the consumable item or consumable items.

36. A container for accommodating a plurality of unarranged consumable items for use with the method according to claim 23, wherein a material for the container is plastic, wherein the container is designed as a bag which after filling with the consumable items and subsequent treatment is sealed and stored in a subsequently sealed outer packaging and wherein a treatment carrier, treated with an appropriate quantity of a sterilisation agent, is introduced into the container.

37. The container according to claim 36, wherein the plastic material has a set permeability so that condensation of the treatment agent inside the container is prevented.

38. The container according to claim 37, wherein a plastic bag in polyethylene is provided as the container.

39. The container according to claim 37, wherein a plastic bag in polyamide is provided as the container.

40. The container according to claim 36, wherein the bag is folded sealed at its open end.

41. An outer packaging for accommodating one or more container(s) for use with the method according to claim 23, wherein the material for the outer packaging cardboard is used, so that an outer carton with closable flaps results, which are folded down and suitably secured in order to close the outer carton and wherein the outer carton is partially wetted on its inside with a treatment agent or a treatment carrier, treated with a suitable quantity of a sterilisation agent, is introduced into the outer carton.
